SEQUENCE LISTING (4 sequences)

SEQ ID NO: 1 - protein, 109 aa, Bos taurus
CELTNITITVEKEECGFCISINTTWCAGYCYTRDLVYRDPARPNIQKTCTFKELVYETVKVPGCAHHADSLYTYPVATECHCSKCDSDSTDCTVRGLGPSYCSFREIKE

SEQ ID NO: 2 - protein, 96 aa, Bos taurus
FPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 3 - protein, 253 aa, synthetic construct
MKSVQFCFLFCCWRAICCRSCELTNITITVEKEECGFCISINTTWCAGYCYTRDLVYRDPARPNIQKTCTFKELVYETVKVPGCAHHADSLYTYPVATECHCSKCDSDSTDCTVRGLGPSYCSFREIKESSSSKAPPPSLPSPSRLPGPSDTPILPQFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 4 - DNA, 762 bp, synthetic construct
  source 1..762: mol_type = other DNA
atgaagtctgtccagttctgtttccttttctgttgctggagagcaatctgctgcagaagctgcgagctgaccaacatcaccatcacggtggagaaagaggaatgtggcttctgcataagcatcaacaccacgtggtgtgcaggctactgctacacccgggacttggtgtacagggacccagcaaggcccaatatccagaaaacgtgtaccttcaaggagctggtctacgagacggtgaaagtgcctggctgtgctcaccatgcagactccctgtacacgtacccagtagccactgaatgtcactgcagcaagtgcgacagcgacagcactgactgcaccgtgagaggcctggggcccagctactgctccttcagggaaatcaaagaatcctcttcctcaaaggcccctcccccgagccttccaagtccatcccgactcccggggccctcggacaccccgatcctcccacaatttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatcctaa

(19) United States
(12) Patent Application Publication
(10) Pub. No.: US 2025/0222073 A1
(43) Pub. Date: Jul. 10, 2025

(54) STIMULATION OF SUPEROVULATION IN BOS TAURUS

(71) Applicant: CEVA SANTE ANIMALE, LIBOURNE (FR)

(72) Inventors: Mark COLGIN, LIBOURNE (FR); Naomi ISAKA, LIBOURNE (FR); Alessio VALENZA, LIBOURNE (FR)

(21) Appl. No.: 18/852,932

(22) PCT Filed: Mar. 31, 2023
(86) PCT No.: PCT/EP2023/058460

§ 371 Date: Sep. 30, 2024

(30) Foreign Application Priority Data
Apr. 1, 2022 (EP) 22166400.6

(51) Int. Cl. A61K 38/24; A61D 19/02; A61P 15/08

(52) U.S. Cl. CPC A61K 38/24; A61D 19/02; A61P 15/08

(57) ABSTRACT

The present invention relates to methods and compositions for increasing superovulation in Bos taurus cows using Follicle Stimulating Hormone.

The present invention relates to methods and compositions for inducing, causing or stimulating superovulation in Bos taurus cows. The invention also relates to methods and compositions for increasing insemination rate in Bos taurus cows. The invention relates to methods and compositions for ovum pick-up in Bos taurus cows. The invention may be used in artificial insemination or in vitro fertilization.

### BACKGROUND OF THE INVENTION

The ability to increase reproductive performance in non-human mammals such as cattle, horses or other ungulates can have a significant benefit to owners. This can be achieved through increasing fertility and/or fecundity in such non-human mammals. Currently, several methods or protocols are proposed to increase reproductive performance, based on the use of estradiol and other hormones such as GnRH (Gonadotropin Releasing Hormone), LH (Luteinizing Hormone), FSH (Follicle stimulating Hormone), or progestagens (such as progesterone) to mimic natural or close to natural levels of hormones occurring at the target specie. Some of these protocols include a step to synchronize ovulation in females to facilitate stimulation, growth and ovulation of follicles in a synchronized fashion allowing the fixed-time artificial insemination, avoiding the necessity of estrus detection. These so-called synchronization protocols are widely used in commercial dairy and beef herds worldwide.

One of the most common treatments used to synchronize the emergence of follicular waves involves the use of different types of estrogens. However, this steroid hormone cannot be used in many parts of the world and alternative methods have been developed to control follicular and luteal phases aiming for instance the improvement of superovulation process of embryo-donor cows in countries where estradiol is not available. An approach that seems to be a promising option is to initiate FSH treatments at the time of the emergence of a new follicular wave, following a GnRH-induced ovulation. In addition, because the half-life of currently available FSH products are generally short (approximately 6 h) superovulatory protocols involve twice a day FSH treatments throughout 4 to 5 days, which are fairly time consuming, stressful and subject to error. Therefore, the standard superovulatory approach is based on multiple injections of FSH at the time of emergence of follicular waves that normally happens 8 to 12 days after naturally occurring estruses. This method is known as “estrus-based”, and while efficient in terms of embryo production, is conditioned by the stage of the estrus cycle and requires estrus detection. Later studies have found that fertilization rates are significantly lower in “estrus-based” programs as compared to modern synchronization protocols that allow a more ideal interval from insemination to ovulation.

Studies have been conducted in order to determine whether lengthening the FSH stimulation protocol could increase the ovulatory response. The authors appear to conclude that extending the FSH injection period from 4 consecutive days to 7 consecutive days could sustain follicle growth and result in more follicles acquiring the capacity to ovulate. More studies are ongoing to confirm this hypothesis in different cattle breeds.

In another reported protocol, cows were treated with FSH during 3 days instead of 4, and the last 2 injections, on day 4, were replaced by 2 eCG (equine Chorionic Gonadotropin) injections (Barros et al., 2008). This treatment appeared to increase the number of embryos, although the time of treatment is still long. In addition, eCG is a fairly long molecule and have been described to induce antibody formation when repeatedly used, and perhaps limiting the wide/frequent use of these types of protocols.

In the attempt to reduce the number of FSH injections, an alternative protocol has been developed wherein FSH is embedded in a slow-release polymer (hyaluronan). Using this slow-release formulation, a single injection was made after “estrus-based” superovulation schedules. However, the authors conclude that the hyaluronan is either too viscous and that the single intramuscular injection resulted in a lower superovulatory response (Bo et al., 2010).

EP2134165 proposes a method for increasing reproduction using a single-chain FSH wherein both subunits are linked covalently by a peptide linker, which is administered in a single dose of 5-150 μg.

WO2014/076231 reports the use of RbFSH analog at a single dose of 50 μg or 100 μg to increase reproductive performance.

### SUMMARY OF THE INVENTION

In one aspect, the present invention relates to methods and compositions and uses for inducing, causing or stimulating superovulation (“SO”) in Bos taurus cows using a one-time Follicle Stimulating Hormone (“FSH”) administration. The invention also relates to such methods and compositions and uses for increasing insemination rate of Bos taurus cows.

In another aspect, the present invention relates to methods and compositions and uses for increasing or improving ovum-pick-up (“OPU”) in Bos taurus cows using a one-time Follicle Stimulating Hormone (“FSH”) administration. The invention also relates to such methods and compositions and uses in artificial insemination or in vitro fertilization methods in Bos taurus cows.

By conducting various tests and research, the inventors have found that in Bos taurus cows, improved ovulation or oocyte production can be obtained using FSH at a particular dosage range and schedule. In particular, by testing efficacy of FSH according to various protocols and in different types of cows, it has been found that the response differs depending on the type of animals and type of effect that is contemplated. More particularly, in Bos taurus cows, contrary to previous reports conducted in other types of cows, it has surprisingly been found that a most efficient superovulation can be obtained with a single administration of a FSH at a dose comprised between 200 μg and 400 μg, most preferably between 250 μg and 350 μg. Such findings are surprising in view of the prior reports of best efficacy at a dosage of about 50 μg. Furthermore, the inventors have also surprisingly found that the best efficacy for allowing or improving Ovum Pick Up in a Bos taurus cow is obtained at a dosage of about 100 μg to 200 μg FSH, which thus differs from the dosage suitable for SO. Accordingly, surprisingly, there is variability between subtypes of cows, and there is variability in a same subtype of cows for best performance in SO or OPU. Identifying the most efficient conditions for inducing SO or OPU in Bos taurus could thus not be extrapolated from the prior art.

In one aspect, the invention stems from the finding that high superovulation can be induced in Bos taurus cows by a single FSH administration at a dose of between 200 μg and 400 μg, in particular between 300 μg and 400 μg. Such finding is surprising in view of earlier reports of best efficacy using much lower dose (e.g., 50 μg) and is view of further data obtained by the inventors showing that most efficient OPU process is achieved in Bos taurus cows with a single dose of about 100 μg.

In another aspect, the invention stems from the finding that high OPU can be induced or achieved in Bos taurus cows by a single FSH administration at a dose of between 100 and 200 μg. Such finding is surprising in view of earlier reports of OPU with no FSH injection, and is view of further data obtained by the inventors showing that most efficient SO is induced in Bos taurus cows with a single dose of about 200-400 μg.

A particular object of the invention relates to the use of a FSH, or of a composition comprising a FSH, to induce, cause or stimulate superovulation in a Bos taurus cow, wherein said FSH is administered once during estrous cycle to said Bos taurus cow at a dose comprised between 200 μg and 400 μg, in particular between 300 μg and 400 μg.

Another object of the invention resides in a method for inducing or stimulating or increasing superovulation in a Bos taurus cow, comprising administering a FSH to said Bos taurus cow once during estrous cycle at a dose comprised between 200 μg and 400 μg, in particular between 300 μg and 400 μg.

Another object of the invention resides in a FSH for use in a method for inducing or stimulating or increasing superovulation in a Bos taurus cow, said method comprising administering said FSH to said Bos taurus cow once during estrous cycle at a dose comprised between 200 μg and 400 μg, in particular between 300 μg and 400 μg.

A further particular object of the invention relates to the use of a FSH, or of a composition comprising a FSH, in a method of ovum pick-up Bos taurus cow, wherein said FSH is administered to said Bos taurus cow at a dose comprised between 100 μg and 200 μg. Preferably, FSH is administered only once and is followed by oocyte collection.

A further preferred object of the invention relates to the use of a FSH, or of a composition comprising a FSH, in a method of ovum pick-up Bos taurus cow, wherein said FSH is administered once during estrous cycle to said Bos taurus cow at a dose comprised between 100 μg and 200 μg. Preferably, the method further comprises a subsequent step of collecting oocytes.

Another object of the invention resides in a method for ovum pick up in a Bos taurus cow, comprising administering a FSH to said Bos taurus cow at a dose comprised between 100 μg and 200 μg. Preferably, FSH is administered only once and is followed by oocyte collection.

Another preferred object of the invention resides in a method for ovum pick up in a Bos taurus cow, comprising administering a FSH to said Bos taurus cow once during estrous cycle at a dose comprised between 100 μg and 200 μg. Preferably, the method further comprises a subsequent step of collecting oocytes.

Another object of the invention resides in a FSH for use in a method for ovum pick up in a Bos taurus cow, said method comprising administering said FSH to said Bos taurus cow at a dose comprised between 100 μg and 200 μg. Preferably, FSH is administered only once and is followed by oocyte collection.

Another preferred object of the invention resides in a FSH for use in a method for ovum pick up in a Bos taurus cow, said method comprising administering said FSH to said Bos taurus cow once during estrous cycle at a dose comprised between 100 μg and 200 μg. Preferably, the method further comprises a subsequent step of collecting oocytes.

Preferably, the FSH is a recombinant FSH (IFSH).

As will be disclosed further, in a preferred embodiment, the FSH comprises a bovine FSH alpha subunit and a bovine FSH beta subunit covalently linked to each other by a peptide linker.

The invention may be used in any Bos taurus cow, in particular any mature Bos taurus cow, such as young adults or older animals, dairy or beef.

### LEGEND TO THE FIGURES

FIG. 1: Nucleotide and amino acid sequence of a FSH for use in the invention. The CTP linker sequence which links the beta and alpha subunits is underlined. The underlined and bold nucleotides encode the signal peptide.

FIG. 2: Graphic showing the number of small (<6 mm), medium (6-10 mm) and large (>10 mm) follicles in heifer donors (Holstein) submitted to OPU with different doses of IFSH for superstimulation.

FIG. 3: Graphic showing the number of small (<6 mm), medium (6-10 mm) and large (>10 mm) follicles in lactating Holstein donors submitted to OPU with different doses of rFSH for superstimulation.

### DETAILED DESCRIPTION OF THE INVENTION

The present invention provides methods to induce or improve SO or OPU in Bos taurus cows. The invention uses a FSH, in a single shot per cycle schedule, under conditions that can most efficiently induce or increase superovulation, quality embryo production, quality oocyte production, pregnancy rate and/or OPU.

### Definitions

The term “superovulation” is generally characterized by a high/increased number of ovulated follicles and/or of fertile ova as compared to a regular ovulation. Typically, within the context of the invention, the term superovulation is characterized by the induction, per Bos taurus cow, of at least 5 (preferably at least 6) freezable embryos (grade 1+2+3) with a percentage of embryo viability above 45%, preferably above 50%, and at least 80% ovulating cows. Most preferred superovulation according to this invention is characterized by at least 6 freezable embryos (grade 1+2+3) per Bos taurus cow, with a percentage of embryo viability above 50%, and at least 80% ovulating cows, more preferably at least 100% ovulating cows.

The term “Ovum pick-up” designates a protocol comprising a step of recovering oocytes from a cow, typically with the aim of performing artificial fertilization or fecundation. For of OPU achievement (and suitable subsequent fertilization/fecundation), it is desirable to collect quality oocytes with most likelihood of being fertilizable into quality embryos. Within the context of the invention, improved OPU is preferably characterized by a recovery rate of oocytes of at least 60%, preferably at least 65% and a level of large follicles of at least 3%, preferably at least 4%, at least 5%, or at least 6%. Preferably, a recovery rate of oocytes of at least 60%, preferably at least 65% is obtained and (i) less than 50% follicles are small follicles, more preferably less than 40%, or even less than 35%, and/or (ii) more than 40% follicles are medium follicles, preferably more than 50%, and/or (iii) more than 5% follicles are large follicles, preferably more than 6%. Most preferred OPU is achieved where the percentage of small vs medium vs large follicles is as follows: large: >6%, medium: >50%; small: <35%.

The term “fertility” or “fertile” refers, within the context of this invention, to the ability to produce fertilizable oocytes.

The term “fecund” or “fecundity” refers, within the context of this invention, to the ability to complete a pregnancy.

The term “pregnant” refers to a non-human mammal or to a group of non-human mammals some of which being currently pregnant or that has been inseminated and may be pregnant.

As used herein, the term “estrus” refers to the period during which a non-human mammal is most likely to become pregnant. Estrus may be detected or monitored by behavioral demonstration that a non-human mammal is in heat, including showing standing heat.

“Insemination” refers to introducing semen by any method known in the art, including, but not limited to, natural and Artificial Insemination (AI) and in vitro fertilization (IVF).

A “group” of animals designates any group of at least 2 non-human mammals, such as a herd or flock. A typical group contains between 2 and 5000 animals.

The term “administration” refers to all route of administration such as oral, enteral mucosal, parenteral or percutaneous. Preferably the administration route is an injection.

The term “follicle synchronization” refers, within the context of this invention, to synchronization of the emergence of follicular waves.

The term “Bos taurus” refers to the taurus subtype of cows. Such subtype is also referred to as Bos taurus taurus. Bos taurus is a different subtype of cows than Bos indicus (also referred to as Bos taurus indicus).

Examples of Bos taurus breeds include Red Angus, Black Angus, Holstein, Friesian, Charolais, Limousin, Wagyu, Simmental, Jersey, Brown swiss, Hereford/Angus, cross and Crossbred, for instance.

Examples of Bos indicus include Nelore, Afrikaner, Bachaur cattle, Boran, Deoni, Feng Catle, Gaolao, Gir, Guzera, Indo-Brazillian, Malvi, Nagori, Nimari, cross and Crossbred, for instance.

The term “dry cow” refers to a dairy cow that is in a stage of its lactation cycle where milk production ceases prior to calving.

### FSH

Follicle Stimulating Hormone belongs to the class of fertility hormones. FSH is composed of two chains (or subunits), termed alpha and beta. Under physiological conditions, both subunits are linked by non-covalent interaction. The nucleotide and amino acid sequences of FSH from various species have been disclosed and are available in public databases such as bovine FSH (Kim K E et al., 1988, “nucleotide sequence of the bovine gene for follicle-stimulating hormone beta-subunit”, DNA 1988, 7 (4): 227-233) or human FSH: gene bank number: CAA43996.1.

The present invention preferably utilizes a recombinant FSH (rFSH), e.g., a FSH produced by recombinant technology and/or engineered using recombinant techniques.

In a particular embodiment, the FSH is a recombinant bovine FSH or a variant thereof. Variants refer to FSH having from 1 to 10, preferably 1 or 2 or 3 amino acid modifications (e.g., substitution, addition, deletion) as compared to a reference sequence, which modification(s) do not substantially alter the activity of FSH. Such modification(s) may be performed in order to e.g., improve the half-life of FSH.

In a preferred embodiment, the FSH used in the invention is a FSH having an alpha subunit and a beta subunit covalently linked together. Such preferred FSH may also be designated “single chain” FSH.

In a more preferred embodiment, the invention uses a recombinant FSH in which an alpha subunit is covalently linked to a beta subunit by a peptide linker. Preferably, the alpha subunit is an alpha subunit of a bovine FSH (or a variant thereof), and/or the beta subunit is a beta subunit of a bovine FSH (or a variant thereof). The peptide linker may be any peptide linker which does not substantially affect the conformation or activity of FSH. In a preferred embodiment, the linker is a CTP linker, e.g., a linker which comprises a sequence of the carboxy terminal peptide of human chorionic gonadotropin as described e.g., in U.S. Pat. No. 6,242,580 and/or US2008/0312151. In another preferred embodiment, the linker is a peptide comprising the (G₄S) sequence, which may be repeated once or several times.

A particular example of an alpha subunit of a bovine FSH is represented in SEQ ID NO: 1. Said sequence, as well as variants thereof, such as a variant with 1 or 2 or 3 amino acid modifications (e.g., substitution, addition, deletion) which do not substantially alter the activity of FSH, are suitable for use in the present invention.

A particular example of a beta subunit of a bovine FSH is represented in SEQ ID NO: 2. Said sequence, as well as variants thereof, such as a variant with 1 or 2 or 3 amino acid modifications (e.g., substitution, addition, deletion) which do not substantially alter the activity of FSH, are suitable for use in the present invention.

In a preferred embodiment, the FSH for use in the present invention has a half-life in a physiological solution of at least 15, preferably at least 20 hours.

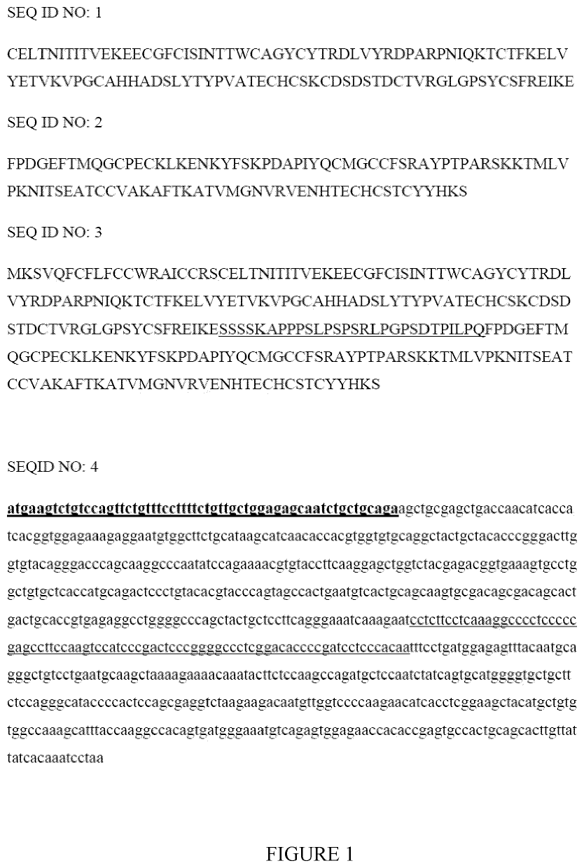

The cDNA and amino acid sequences of a particular recombinant FSH for use in the present invention are provided in FIG. 1 (SEQ ID NO: 3 and SEQ ID NO: 4). The first part (amino acids 1-129) of SEQ ID NO:3 corresponds to the sequence of a bovine FSH beta subunit (SEQ ID NO: 2), the central part corresponds to a carboxy terminal peptide linker (amino acids 130-157), and the third part (amino acids 158-253) corresponds to the sequence of a bovine FSH alpha subunit (SEQ ID NO: 1).

In a particular embodiment, the invention uses a recombinant FSH having, comprising, consisting essentially of, or consisting of (i) the amino acid sequence of SEQ ID NO: 3, or (ii) an amino acid sequence having at least 90% identity to amino acid sequence SEQ ID NO: 3 over the entire length thereof, preferably 95% or more.

The FSH is preferably isolated, more preferably essentially pure, e.g., with a purity level of at least 95%, more preferably at least 97, 98 or 99%.

In a particular embodiment, the FSH or recombinant FSH is used in a composition comprising a suitable pharmaceutical formulation. The pharmaceutical formulation may comprise one or several excipients or carriers, such as excipients suitable for a liquid or solid formulation. The formulation is typically a liquid formulation, such as a solution or suspension, preferably adapted for injection. The liquid formulation may contain any suitable diluent, such as a saline solution, a buffered solution, a physiological solution, etc. Examples of such suitable diluents include phosphate buffered saline. Additives may be present in the formulation, such as preservatives, stabilizers, etc. In a particular embodiment, the formulation is a liquid formulation at a dosage of about 50 μg FSH/ml.

### Superovulation

The invention relates to methods for inducing/increasing/stimulating superovulation, and/or for increasing pregnancy rate in (a) Bos taurus cow(s), using FSH.

As discussed above, in one aspect, the invention stems from the finding that high superovulation can be induced in a Bos taurus cow by a single FSH administration at a dose of between 200 μg and 400 μg. Such finding is surprising in view of earlier reports of best efficacy using much lower dose in other types of cows (e.g., 50 μg) and in view of further data contained in this application showing that most efficient OPU process is achieved in Bos taurus cows with a single dose of about 100 μg.

A particular object of the invention thus relates to the use of a FSH, or of a composition comprising a FSH, to induce, cause or stimulate superovulation in a Bos taurus cow, wherein said FSH is administered once during estrous cycle to said Bos taurus cow at a dose comprised between 200 μg and 400 μg, in particular between 300 μg and 400 μg.

Another object of the invention resides in a method for inducing or stimulating or increasing superovulation in a Bos taurus cow, comprising administering a FSH to said Bos taurus cow once during estrous cycle at a dose comprised between 200 μg and 400 μg, in particular between 300 μg and 400 μg.

Another object of the invention resides in a FSH for use in a method for inducing or stimulating or increasing superovulation in a Bos taurus cow, said method comprising administering said FSH to said Bos taurus cow once during estrous cycle at a dose comprised between 200 μg and 400 μg, in particular between 300 μg and 400 μg.

In a preferred embodiment, which may be combined with any other particular and/or preferred embodiment, the FSH is administered at a dose of 200 μg, 220 μg, 230 μg, 250 μg, 260 μg, 270 μg, 280 μg, 290 μg, 300 μg, 310 μg, 320 μg, 330 μg, 340 μg, 350 μg, 370 μg, or 400 μg, preferably at a dose comprised between 250 μg and 350 μg, even more preferably at a dose of 260 μg, 270 μg, 280 μg, 290 μg, 300 μg, 310 μg, 320 μg, 330 μg, or 340 μg, or at a dose comprised between 300 μg and 400 μg, such as at a dose of 350 μg, 360 μg, 370 μg, 380 μg, 390 μg.

Data presented in the present application show that such dosage range causes ovulation of above 80% cows, typically in 100% cows, and can induce a high number of freezable viable embryos, thus allowing improved insemination/pregnancy rate.

In a particular embodiment, which may be combined with any other particular and/or preferred embodiment, the FSH is administered once between day 7 and day 12 of estrous cycle, preferably between day 8 and day 11, such as at Day 8, Day 9, Day 10 or Day 11 of estrous cycle, even more preferably at day 9 or day 10. Estrous cycle can easily be detected and/or monitored by the skilled person in the art. In some instances, if appropriate, ultrasound technique may be used to determine the onset of estrous cycle, according to methods well known in the art (see also experimental section).

In a particular embodiment, the invention relates to a method or use comprising administering FSH to the Bos taurus cow, said FSH being administered once between day 7 and day 12 of estrous cycle of said cow at a dose comprised between about 200 μg and about 400 μg, preferably by injection, more preferably by intramuscular injection.

In a further particular embodiment, which may be combined with any other particular and/or preferred embodiment, the FSH is administered to synchronized cows (e.g., to cows that have been previously treated to synchronize follicles). Methods of synchronizing cow are known per se in the art, as discussed in a further section below.

FSH, or the composition comprising FSH, may be administered by any suitable route and with any suitable means. Preferably, FSH is administered through systemic administration, particularly injection, such as intramuscular, intravenous, or subcutaneous injection. A preferred administration route is intramuscular injection.

In a preferred embodiment, the composition or method or protocol of the invention uses a single dose of FSH of between 250 μg and 350 μg, preferably in a liquid formulation suitable for injection, preferably intramuscular injection.

In another preferred embodiment, the composition or method or protocol of the invention uses a single dose of FSH of about 300 μg, preferably in a liquid formulation suitable for injection, preferably intramuscular injection.

The results presented in the experimental section show that such a single administration causes production of high number fertile transferable embryos.

In a further particular embodiment, which may be combined with any other particular and/or preferred embodiment, the methods, uses, or protocols of the invention further comprise a step of inseminating the cow(s).

In that respect, an object of the invention resides in a method for inseminating a Bos taurus cow, comprising (i) inducing or stimulating superovulation according to the invention and (ii) inseminating the cow.

In a particular embodiment, the invention relates to a method or use comprising:
(a) administering FSH to a Bos taurus cow, said FSH being administered once between day 7 and day 12 of estrous cycle of said cow at a dose comprised between about 200 μg and about 400 μg, in particular between 300 μg and 400 μg, preferably by injection, more preferably by intramuscular injection; and
(b) inseminating the cow, preferably by artificial insemination.

Preferably, insemination is performed or allowed to occur near the time of ovulation. In a preferred embodiment, insemination is performed (or allowed to occur) between 3 and 8 days, more preferably between 4 to 7 days after injection of FSH of step (a). Preferably, insemination is performed by artificial insemination.

One or more additional hormones, such as luteinizing hormone, chorionic gonadotropin and/or prostaglandin, can optionally be administered to further ameliorate insemination. In one embodiment, a prostaglandin is administered to the cow(s), in addition to administration of FSH. The prostaglandin may be administered as a single dose, typically by injection, or as multiple doses, administered several hours apart. In one embodiment, one dose is administered, comprised between 5 and 50 mg. The prostaglandin is preferably administered at day 2, 3 or 4 after FSH administration.

In another embodiment, a GnRH is administered to the cow(s), in addition to administration of FSH (and optionally a prostaglandin). GnRH is administered as a single dose or, typically, in two sequential doses which may be administered several hours (or 1 day) apart. In one embodiment, the method/use comprises administering GnRH to said cow(s), preferably at day 4, 5 or 6 after FSH administration, even more preferably at days 5 and 6 after FSH administration. The total dose of GnRH is preferably between 50 μg and 200 μg. If two sequential administrations are performed, it is preferred to administer 100 μg each time. GnRH is preferably administered by injection(s).

In a particular embodiment, which may be combined with any other particular and/or preferred embodiment, the methods, uses, or protocols of the invention thus further comprises administering a prostaglandin to said cow, preferably at day 2, 3 or 4 after FSH administration.

In a further particular embodiment, which may be combined with any other particular and/or preferred embodiment, the methods, uses, or protocols of the invention further comprise administering GnRH to said cow, preferably at day 4, 5 or 6 after FSH administration, even more preferably at days 5 and 6 after FSH administration.

In this respect, in a particular embodiment, the invention relates to a method or use or protocol as described above, comprising:
(a) administering FSH to a Bos taurus cow, said FSH being administered once between day 7 and day 12 of estrous cycle of said cow at a dose comprised between about 200 μg and about 400 μg, in particular between 300 μg and 400 μg, preferably by injection, more preferably by intramuscular injection;
(b) administering a prostaglandin and/or a GnRH to said cow; and
(c) inseminating the cow, preferably by artificial insemination.

As discussed above, the FSH used in the invention preferably comprises a bovine FSH alpha subunit and a bovine FSH beta subunit covalently linked to each other by a peptide linker. In a particular embodiment, the invention uses a FSH having, comprising, consisting essentially of, or consisting of (i) the amino acid sequence of SEQ ID NO: 3, or (ii) an amino acid sequence having at least 90% identity to amino acid sequence SEQ ID NO: 3 over the entire length thereof, preferably 95% or more, and retaining a FSH activity or profile as defined above.

In a further particular embodiment, the method/use/protocol comprises:
(a) injecting FSH to a Bos taurus cow, said FSH comprising a bovine FSH alpha subunit and a bovine FSH beta subunit covalently linked to each other by a peptide linker, said FSH being administered once between day 8 and day 11 of estrous cycle of said cow at a dose comprised between about 200 μg and about 400 μg, in particular between 300 μg and 400 μg, more preferably by intramuscular injection;
(b) optionally administering a prostaglandin and/or a GnRH to said cow; and
(c) inseminating the cow 3 to 8 days, more preferably 4 to 7 days after injection of FSH of step (a), preferably by artificial insemination.

In a particular embodiment, the method/use/protocol is performed on a group of synchronized cows (e.g., a group of cows that have been treated to synchronize follicles). Such embodiment can avoid the need to detect or monitor estrus.

The uses/methods/protocols of the invention as defined above can be implemented during one, several or each estrus cycle of the Bos taurus cow(s).

### Ovum Pick-Up

In another aspect, the invention stems from the finding that high OPU can be induced or achieved in Bos taurus cows by a single FSH administration at a dose of between 100 and 200 μg. Such finding is surprising in view of earlier reports of OPU with no FSH injection, and is view of further data presented in this application showing that most efficient SO is induced in Bos taurus cows with a single dose of about 200-400 μg. In particular, the inventors have shown that high OPU can be induced or achieved in Bos taurus heifers by a single FSHadministration at a dose of 100 μg. The inventors have further shown that high OPU can be induced or achieved in Bos taurus lactating cows by a single FSHadministration at a dose of 150 μg.

A particular object of the invention thus relates to the use of a FSH, or of a composition comprising a FSH, in a method of ovum pick-up Bos taurus cow, wherein said FSH is administered to said Bos taurus cow at a dose comprised between 100 μg and 200 μg. Preferably, FSH is administered only once and is followed by oocyte collection.

The present invention further relates to the use of a Follicle Stimulating Hormone (FSH) for the manufacture of a treatment for ovum pick-up application in a Bos taurus cow, wherein said FSH is administered to said Bos taurus cow at a dose comprised between 100 μg and 200 μg, between 100 μg and 150 μg, in particular at 100 μg or 150 μg.

A preferred object of the invention thus relates to the use of a FSH, or of a composition comprising a FSH, in a method of ovum pick-up Bos taurus cow, wherein said FSH is administered once during estrous cycle to said Bos taurus cow at a dose comprised between 100 μg and 200 μg. The method further comprises a subsequent step of collecting oocytes.

Another preferred object of the invention relates to the use of a FSH, or of a composition comprising a FSH, in a method of ovum pick-up Bos taurus cow, wherein said FSH is administered once during estrous cycle to said Bos taurus cow at a dose of 100 μg or 150 μg. The method further comprises a subsequent step of collecting oocytes.

Another object of the invention resides in a method for ovum pick up in a Bos taurus cow, comprising administering a FSH to said Bos taurus cow at a dose comprised between 100 μg and 200 μg. Preferably, FSH is administered only once and is followed by oocyte collection.

Another object of the invention resides in a method for ovum pick up in a Bos taurus cow, comprising administering a FSH to said Bos taurus cow at a dose of 100 μg or 150 μg. Preferably, FSH is administered only once and is followed by oocyte collection.

Another preferred object of the invention resides in a method for ovum pick up in a Bos taurus cow, comprising administering a FSH to said Bos taurus cow once during estrous cycle at a dose comprised between 100 μg and 200 μg. The method further comprises a subsequent step of collecting oocytes.

Another preferred object of the invention resides in a method for ovum pick up in a Bos taurus cow, comprising administering a FSH to said Bos taurus cow once during estrous cycle at a dose of 100 μg or 150 μg. The method further comprises a subsequent step of collecting oocytes.

Another object of the invention resides in a FSH for use in a method for ovum pick up in a Bos taurus cow, said method comprising administering said FSH to said Bos taurus cow at a dose comprised between 100 μg and 200 μg. Preferably, FSH is administered only once and is followed by oocyte collection.

Another object of the invention resides in a FSH for use in a method for ovum pick up in a Bos taurus cow, said method comprising administering said FSH to said Bos taurus cow at a dose of 100 μg or 150 μg. Preferably, FSH is administered only once and is followed by oocyte collection.

Another preferred object of the invention resides in a FSH for use in a method for ovum pick up in a Bos taurus cow, said method comprising administering said FSH to said Bos taurus cow once during estrous cycle at a dose comprised between 100 μg and 200 μg. The method further comprises a subsequent step of collecting oocytes.

Another preferred object of the invention resides in a FSH for use in a method for ovum pick up in a Bos taurus cow, said method comprising administering said FSH to said Bos taurus cow once during estrous cycle at a dose of 100 μg or 150 μg. The method further comprises a subsequent step of collecting oocytes.

In a preferred embodiment, which may be combined with any other particular and/or preferred embodiment, the FSH is administered at a dose of 100 μg, 110 μg, 120 μg, 130 μg, 140 μg, 150 μg, 160 μg, 170 μg, 180 μg, 190 μg, or 200 μg, preferably at a dose comprised between 100 μg and 150 μg, even more preferably at a dose of about 100 μg. In a particular embodiment, the FSH is administered at a dose of 100 μg. In another particular embodiment, the FSH is administered at a dose of 150 μg.

Data presented in the present application show that such dosage range induce high % of large follicles with a high oocyte recovery rate, thus allowing improved fertilization.

By conducting specific works, the inventors have discovered that the administration of a single dose of 100 μg is particularly efficient for OPU in Bos taurus heifers. Similarly, the inventors have discovered that the administration of a single dose of 150 μg is particularly efficient for OPU in lactating Bos taurus cows.

Estrous cycle can easily be detected and/or monitored by the skilled person in the art. In some instances, if appropriate, ultrasound technique may be used to determine the onset of estrous cycle, according to methods well known in the art (see also experimental section).

In a particular embodiment, the invention relates to a method or use for OPU comprising administering FSH to the Bos taurus cow, said FSH being administered once at a dose comprised between about 100 μg and about 200 μg, preferably by injection, more preferably by intramuscular injection.

In preferred embodiment, the invention relates to a method or use for OPU comprising administering FSH to a Bos taurus heifer, said FSH being administered once at a dose of 100 μg, preferably by injection, more preferably by intramuscular injection.

In another preferred embodiment, the invention relates to a method or use for OPU comprising administering FSH to a lactating Bos taurus cow, said FSH being administered once at a dose of 150 μg, preferably by injection, more preferably by intramuscular injection

FSH, or the composition comprising FSH, may be administered by any suitable route and with any suitable means. Preferably, FSH is administered through systemic administration, particularly injection, such as intramuscular, intravenous, or subcutaneous injection. A preferred administration route is intramuscular injection.

In a preferred embodiment, the composition or method or protocol of the invention uses a single dose of FSH of between 100 μg and 200 μg, preferably in a liquid formulation suitable for injection, preferably intramuscular injection.

In another preferred embodiment, the composition or method or protocol of the invention uses a single dose of FSH of 100 μg, preferably in a liquid formulation suitable for injection, preferably intramuscular injection.

In another preferred embodiment, the composition or method or protocol of the invention uses a single dose of FSH of 150 μg, preferably in a liquid formulation suitable for injection, preferably intramuscular injection.

The results presented in the experimental section show that such a single administration causes production of high number high fecundity oocytes.

In a further particular embodiment, which may be combined with any other particular and/or preferred embodiment, the OPU methods, uses, or protocols of the invention further comprise a step of collecting oocytes.

In that respect, an object of the invention resides in a method for obtaining oocytes from a Bos taurus cow, comprising (i) administering FSH to said Bos taurus cow once at a dose comprised between 100 μg and 200 μg, preferably by injection, more preferably by intramuscular injection; and (ii) collecting oocytes.

In a particular embodiment, the Bos taurus cow is a heifer and the dose of FSH is 100 μg.

In another particular embodiment, the Bos taurus cow is a lactating cow and the dose of FSH is 150 μg.

Preferably, oocyte collection is performed at day 2, day 3, day 4 or day 5 after FSH administration. Data obtained by the inventors show that with the method of the invention, a high recovery rate is obtained, above 60%, more preferably above 65%, with high quality oocytes (large follicles representing more than 3% total follicle, preferably more than 4%, more than 5%, even more preferably more than 6%). Such performance allows successful fertilization of collected oocytes.

In a further particular embodiment, which may be combined with any other particular and/or preferred embodiment, the OPU methods, uses, or protocols of the invention further comprise a step of fecundation of collected oocytes, typically in vitro fecundation (FIV).

In that respect, an object of the invention resides in a method comprising (i) administering FSH to a Bos taurus cow once at a dose comprised between 100 μg and 200 μg, preferably by injection, more preferably by intramuscular injection; (ii) collecting oocytes from the cow, preferably at day 2, day 3, day 4 or day 5 after FSH administration; and (iii) fecundating the oocytes, preferably in vitro.

In particular, an object of the invention resides in a method comprising (i) administering FSH to a Bos taurus heifer once at a dose of 100 μg, preferably by injection, more preferably by intramuscular injection; (ii) collecting oocytes from the cow, preferably at day 2, day 3, day 4 or day 5 after FSH administration; and (iii) fecundating the oocytes, preferably in vitro.

Another particular object of the invention resides in a method comprising (i) administering FSH to a Bos taurus lactating cow once at a dose of 150 μg, preferably by injection, more preferably by intramuscular injection; (ii) collecting oocytes from the cow, preferably at day 2, day 3, day 4 or day 5 after FSH administration; and (iii) fecundating the oocytes, preferably in vitro.

In vitro fecundation can be performed at any time after collection. However, it is preferred to perform fecundation between 6 hours and 3 days post collection.

Alternatively, oocytes may be frozen and subsequently thawed for fecundation. Fecundated oocytes may then by cultured in vitro (until embryo develop), or inseminated, according to methods known in the art.

As discussed above, the FSH used in the invention preferably comprises a bovine FSH alpha subunit and a bovine FSH beta subunit covalently linked to each other by a peptide linker. In a particular embodiment, the above methods, uses, or protocols of the invention use a FSH having, comprising, consisting essentially of, or consisting of (i) the amino acid sequence of SEQ ID NO: 3, or (ii) an amino acid sequence having at least 90% identity to amino acid sequence SEQ ID NO: 3 over the entire length thereof, preferably 95% or more, and retaining a FSH activity or profile as defined above.

In a further particular embodiment, the method/use/protocol comprises:
(a) injecting FSH to a Bos taurus cow, said FSH comprising a bovine FSH alpha subunit and a bovine FSH beta subunit covalently linked to each other by a peptide linker, said FSH being administered once to said cow at a dose comprised between about 100 μg and about 200 μg, more preferably by intramuscular injection; and
(b) collecting oocytes from said cow, preferably between 2 and 5 days after FSH administration.

In a particular embodiment, the Bos taurus cow is a heifer and the FSH dose is 100 μg. In another particular embodiment, the Bos taurus cow is a lactating cow and the FSH dose is 150 μg.

In a particular embodiment, the method comprises a further step (c) of fecundating collected oocytes in vitro.

### Follicle Synchronization

In a particular embodiment, the methods/uses/protocols of the invention are performed with synchronized cows. Synchronizing emergence of a new follicular wave, in combination with a FSH treatment of the invention, leads to improved embryo production. Furthermore, by synchronizing follicles, the need to monitor estrus cycle is avoided.

Follicular growth is not continuous in cows, but occurs in waves (2 to 4 waves per cycle). Each wave begins approximately when the dominant follicle in the previous wave gains maximal size, at which time numerous small follicles begin a period of rapid growth. From this group of follicles, one follicle is allowed to grow to a much larger size than the others. This large follicle is called the dominant follicle, because it has the ability to regulate and restrict the growth of the smaller follicles, called subordinate follicles. A few days after reaching maximum size, the dominant follicle begins to regress and die. As the dominant follicle degenerates, its ability to restrict the other follicles is reduced; therefore, a new follicular wave is initiated. A consequence of this dynamic process is that follicles of all sizes, including at least one large follicle, exist on each day of the estrous cycle. Follicle wave synchronization gives the opportunity to treat all cows in a limited period of time and, therefore, to capture the economic benefits of insemination. Upon synchronization of the estrous cycle, a high percentage of treated females show a fertile, closely synchronized estrus and ovulation.

In a particular embodiment, follicle synchronization is obtained by hormonal treatment, preferably with progestagens, progestagen-prostaglandin combinations, prostaglandins alone, progestagen-estrogen combinations, or gonadotropin-prostaglandin combinations with or without progestagens.

In a particular embodiment, follicle synchronization is obtained by one of the following treatments:
PGF2alpha or its analogs;
GnRH+PGF2alpha
A Progestagen (such as progesterone), optionally in combination with estrogen or PGF2alpha or GnRH.

Specific dosages and/or protocols are disclosed in the art such as, e.g., in Thatcher et al., 2001 (American Association of Bovine Practitioner, AABP, Vancouver, 95-105); Diskin et al., 2001 (occasional publication no 26, p 175, British society of Animal Science); or Pursley et al., 1995 (Theriogenology 44 p 915). Also, progestagens may be administered using specific devices such as implants (e.g., PRID, of Ceva Santé Animale).

In another embodiment, follicle synchronization can be obtained by follicular ablation. Ablation of follicle designates the elimination, removal or destruction of at least one follicle. Follicular ablation refers to physical methods of follicular ablation, such as cautery or ultrasound-guided transvaginal follicle aspiration at random stages of the estrous cycle (Bergfelt, 1997). Ablation can be directed to all follicles or only to the one or two largest follicles (Baracaldo et al., 2000), to ensure at least the dominant follicle is suppressed.

In a particular embodiment, the methods, uses, protocols of the invention thus can include a prior step of synchronizing follicles in a group of Bos taurus cows, such as in particular by a method comprising treating a Bos taurus cow (or a group of Bos taurus cows) with a progestagen, a progestagen-prostaglandin combination, a prostaglandin, a progestagen-estrogen combination, or a gonadotropin-prostaglandin combination with or without progestogens.

In a particular embodiment, the invention thus relates to methods, uses, protocols comprising:
(a) treating a Bos taurus cow (or a group of Bos taurus cows) with a progestagen, a progestagen-prostaglandin combination, a prostaglandin, a progestagen-estrogen combination, or a gonadotropin-prostaglandin combination with or without progestogens to synchronize follicles; or providing a Bos taurus cow (or a group of Bos taurus cows) which has been treated with a progestagen, a progestagen-prostaglandin combination, a prostaglandin, a progestagen-estrogen combination, or a gonadotropin-prostaglandin combination with or without progestagens, to synchronize follicles;
(b) administering to the synchronized Bos taurus cow (or group of Bos taurus cows) a single dose of between 200 μg and 400 μg of a FSH, in particular between 300 and 400 μg; and
(c) inseminating the cows, preferably near the time of ovulation and, more preferably, 2 to 7 days, even more preferably 4 to 6 days after administration step (b).

Further aspects and advantages of the invention will be disclosed in the following experimental section, which illustrates the claimed invention.

### EXAMPLES

### Example 1: One Dose Treatment with a Bovine Follicle Stimulating Hormone Induces Superovulation in Mature Beef Cows of Bos taurus Subtype

The objective of the study was to evaluate the efficacy, and design an optimal protocol of a bovine follicle stimulating hormone for superovulation in mature beef cows of Bos taurus subtype.

The study was conducted in full accordance to GCP standards, VICH GL9 Guidance for Industry Good Clinical Practice and CVM GFI 56 Guidance for Industry Protocol Development for Clinical Effectiveness and Target Animal Safety Trials. The study was a single blind, parallel study design of five treatment groups. Fifty mature female beef cattle were randomly assigned to the five groups treated with a recombinant single-chain bovine FSH (50 μg, 100 μg, 200 μg, 300 μg and 400 μg; 10 cows per group) and compared to a historical control.

Following treatment on Day 0 (corresponding to Day 9/10 of the estrous cycle), cows were given prostaglandin to lyse corpora lutea on Day 3, given GnRH to induce ovulation on Days 5 and 6, and artificially inseminated on Day 6. Embryos were recovered and evaluated on Day 12. Parameters measured included number and size of follicles and number of corpora lutea on Day 3, number of corpora lutea on Day 12 and number and quality of embryos on Day 12.

### IVP

The FSH used is a recombinant single chain bovine FSH designated RIPAFOLLITROPIN ALFA (BOVINE). RIPAFOLLITROPIN ALFA (BOVINE) is a glycoprotein with a primary structure consisting of the beta subunit of bovine FSH covalently linked to the alpha subunit of bovine FSH. The amino acid sequence of RIPAFOLLITROPIN ALFA (BOVINE) is provided as SEQ ID NO: 3.

The FSH was formulated in a veterinary composition with the following composition:
Active substance: RIPAFOLLITROPIN ALFA (BOVINE)
Quantity: 476 μg
Concentration: 4.46 mg/mL
Excipients: Phosphate buffered saline
Dosage form: Injectable solution
Packaging: 10 ml Glass vials
Storage conditions: Store at 2-8° C. until use

### Treatment Groups and Randomization Procedures

On Day −3, the cows were assessed for normal health by history and physical examination (including heart rate, respiration rate, and body weight). Also at this time, a manual and ultrasound examination was performed to confirm normal reproductive anatomy and the presence of a corpus luteum for each cow. Fifty (50) cycling mature Bos taurus beef cows with normal corpora lutea and that met all enrollment criteria were enrolled, ranging in age from 24 months to 84 months and with a body weight ranging from 1015 pounds (461 kg) to 1380 pounds (627 kg).

Cows were randomized to treatment group using a computer-generated randomization table provided by the Sponsor on Day −1. Enrolled cows were treated with RIPAFOLLITROPIN ALFA (BOVINE) on Day 0 (i.e., Day 9 or 10 of the estrous cycle) with one of the following treatments shown in Table 1:

TABLE 1
Treatment Groups

| | | | Quantity of |
|---|---|---|---|
| Treatment | | | RIPAFOLLITROPIN |
| Group | N | Volume | ALFA (BOVINE) |
| 1 | 10 | 1 ml | 50 μg |
| 2 | 10 | 2 ml | 100 μg |
| 3 | 10 | 4 ml | 200 μg |
| 4 | 10 | 6 ml | 300 μg |
| 5 | 10 | 8 ml | 400 μg |

IVP was administered once intramuscularly on Day 0. It was administered on the right side of the neck using a sterile plastic disposable 10 ml syringe fitted with a sterile disposable 1.5 inch 18 gauge needle.

All 50 cows received an IM injection of 25 mg prostaglandin 72 hours (+/−2 hours) after RIPAFOLLITROPIN ALFA (BOVINE) treatment. At the same time as this prostaglandin injection an ultrasound examination was performed to evaluate the number and sizes of follicles (small=2-3 mm, medium=4-8 mm and large ≥9 mm) and number of corpora lutea on each ovary.

Forty eight (48) hours (+/−2 hours) after prostaglandin treatment, each cow received an injection of 100 μGnRH, intramuscularly. Prostaglandin and GnRH treatments were given in the right hip and the left hip, respectively.

All enrolled cows were observed for estrus starting at approximately 32 hours after prostaglandin treatments. This commenced during the PM (4 pm-6:21 pm; Deviation 1) on Day 4. All cows received GnRH treatment in the AM (7 am-10 am) on Day 5. Two cows were in heat in PM on Day 4 and were artificially inseminated in the AM (7 am-10 am) on Day 5. Those same cows were artificially inseminated in the PM (4 pm-6:07 pm; Deviation 2) on Day 5. Forty-eight cows were artificially inseminated only once on Day 5 in the PM and received their second artificial insemination in the AM (7 am-10 am) of Day 6. The semen was sourced from a single bull (Money Talks—SEK Genetics, Galesburg, KS 66740). On study day 12 (7 days after the first artificial insemination), a manual examination was conducted to determine the CL count on each ovary.

Embryos were recovered (flushed). After embryo recovery on study day 12, cows were treated with prostaglandin to promote resumption of cyclicity. Embryos from individual cows were processed separately throughout evaluation. Embryos were graded according to the standards of the International Embryo Transfer Society (IETS Manual, 4th Edition).

### Results

A total of 50 beef cows (breeds included Red Angus, Black Angus, Hereford/Angus cross and Crossbred) were enrolled in the study. Cow age and body weights by dose group are summarized in Table 2.

TABLE 2
Summary of the cow demographics and ovulation
outcomes (mean and standard deviation)

| | | |
|---|---|---|
| Dose Group | Age (months) | Weight (pounds) |

| (μg) | Mean | SD | Mean | SD |
|---|---|---|---|---|
| 50 | 49.2 | 19.14 | 1211.0 | 114.89 |
| 100 | 45.6 | 15.80 | 1139.5 | 77.44 |
| 200 | 39.6 | 13.91 | 1169.0 | 109.26 |
| 300 | 52.8 | 18.07 | 1182.5 | 79.42 |
| 400 | 43.2 | 10.12 | 1205.0 | 79.23 |

Ovaries were evaluated by ultrasound on D3 prior to treatment with prostaglandin to evaluate follicular growth in response to each dose of RIPAFOLLITROPIN ALFA (BOVINE). Table 3 shows the mean number of follicles observed for each treatment group. There is a dose effect in response to RIPAFOLLITROPIN ALFA (BOVINE) since the mean number of follicles tends to increase with increasing doses of RIPAFOLLITROPIN ALFA (BOVINE). Ovaries were also evaluated on Day 12 to determine the number of corpora lutea produced to estimate the ovulation rate following treatment with GnRH. Table 3 also lists the mean number of corpora lutea for each treatment group. Fewer follicles ovulated in the lowest (50 and 100 μg) dose groups than the higher (200-400 μg) groups. Total ova were counted as part of the embryo evaluation process, also performed on Day 12. Total ova were higher in the higher dose groups.

TABLE 3
Follicular development and ovulation
rates (mean and standard deviation)

| Dose Group | Follicles | | Corpora Lutea | | Total Ova | |
|---|---|---|---|---|---|---|
| (μg) | Mean | SD | Mean | SD | Mean | SD |
| 50 | 14.2 | 6.51 | 1.0 | 0.47 | 0.3 | 0.48 |
| 100 | 19.2 | 5.77 | 3.2 | 2.62 | 2.8 | 2.10 |
| 200 | 29.3 | 11.46 | 12.1 | 7.16 | 14.3 | 10.74 |
| 300 | 26.8 | 10.77 | 14.9 | 7.61 | 14.3 | 11.35 |
| 400 | 38.4 | 12.98 | 13.7 | 9.75 | 18.0 | 6.70 |

Embryos were collected and evaluated on Day 12. Table 4 below lists the mean and standard deviation for two quality grading categories (Grade 1+2 embryos and Grade 1+2+3 embryos).

TABLE 4
Embryo Evaluation (mean and standard deviation)

| Dose Group | Grade 1 + 2 Embryos | | Grade 1 + 2 + 3 Embryos | |
|---|---|---|---|---|
| (μg) | Mean | SD | Mean | SD |
| 50 | 0.1 | 0.32 | 0.1 | 0.32 |
| 100 | 2.7 | 2.06 | 2.7 | 2.06 |
| 200 | 8.6 | 7.56 | 9.3 | 8.29 |
| 300 | 7.5 | 7.40 | 8.2 | 7.63 |
| 400 | 8.3 | 6.33 | 9.3 | 9.46 |

Results of the statistical analysis for Grade 1+Grade 2 embryos are summarized in Table 5. A statistically significant dose effect was detected for transferable (Grade 1+Grade 2) embryos (Table 5). With increasing dose, we observed the number of transferable embryos increased. Based on the pair-wise comparisons, there were no significant differences between the three highest doses (P >0.05) with regard to the number of transferable embryos. Additionally, a statistically significant dose effect was detected for embryo viability (Table 5).

The number of cows ovulating was higher in the dose groups above the 50 μg dose. The mean ovulation rate in the higher (200-400 μg) dose groups had a mean ovulation rate of 93%.

TABLE 5
Summary of the statistical analysis of transferable
(Grade 1 + Grade 2) embryos and embryo viability
(least squares means, mean square error)

| Dose Group | Transferable | Embryo | Number of cows |
|---|---|---|---|
| (μg) | embryos | viability (%) | ovulating |
| 50 | 0.10^{a} | 33.33^{a} | 3/10 |
| 100 | 2.70^{ac} | 96.43^{b} | 7/10 |
| 200 | 9.56^{b} | 62.70^{a} | 8/10 |
| 300 | 7.50^{bc} | 58.89^{a} | 10/10 |
| 400 | 8.30^{b} | 45.93^{a} | 10/10 |
| MSE | 30.0869 | 723.99 | |
| P-value | 0.0014 | 0.0044 | |
| Linear | 0.0004 | 0.7462 | |
| Quadratic | 0.0643 | 0.0068 | |

^{abc}within a column, values with no common letters are significantly different at P < 0.05.

Results of the statistical analysis for Grade 1+Grade 2+Grade 3 embryos are summarized in Table 6. A statistically significant dose effect was detected for transferable (Grade 1+Grade 2+Grade 3) embryos (Table 6). With increasing dose, the number of transferable embryos increased. Based on the pair-wise comparisons, there were no significant differences between the three highest doses (P >0.05) with regard to the number of transferable embryos. Additionally, a statistically significant dose effect was detected for embryo viability (Table 6).

TABLE 6
Summary of the statistical analysis of transferable
(Grade 1 + Grade 2 + Grade 3) embryos and
embryo viability (least squares means, mean square error)

| Dose, | Transferable | Embryo | Number of cows |
|---|---|---|---|
| μg | embryos | viability, % | ovulating |
| 50 | 0.10^{a} | 33.3^{a} | 3/10 |
| 100 | 2.70^{a} | 96.4^{b} | 7/10 |
| 200 | 10.33^{b} | 67.0^{a} | 8/10 |
| 300 | 8.20^{b} | 62.7^{a} | 10/10 |
| 400 | 9.30^{b} | 52.3^{a} | 10/10 |
| MSE | 33.19 | 724.29 | |
| P-value | 0.0007 | 0.0093 | |
| Linear | 0.0002 | 0.9096 | |
| Quadratic | 0.0720 | 0.0070 | |

^{ab}within a column, values with no common letters are significantly different at P < 0.05.

### Conclusions

The objective of this study was to evaluate efficacy of recombinant FSH (doses of 50 μg, 100 μg, 200 μg, 300 μg and 400 μg; n=10 per group) in Bos taurus beef cows. No safety concerns were noted in this study. RIPAFOLLITROPIN ALFA (BOVINE) stimulated follicular development in a dose-dependent manner as evidenced by an increasing number of follicles observed on Day 3. This translated into a similar relationship observed for the total number of ova, which also increased with increased dose. The mean numbers of corpora lutea and corresponding total ova were consistent, indicating efficiency in corpus luteum count and ova/embryo collection. Except for the 50 μg group, there was a high ovulation rate observed.

Endpoints for Grade 1+2 embryos and Grade 1+Grade 2+Grade 3 embryos were evaluated. Grade 1 and 2 embryos are considered freezable and transferrable while Grade 3 embryos are considered transferrable but not freezable. Results were similar regardless of whether the Grade 3 embryos were considered. Treatment groups 3, 4 and 5 (200 μg, 300 μg and 400 μg; respectively) produced a greater number of high quality (Grade 1 and 2) embryos per flush than the groups administered 50 or 100 μg RIPAFOLLITROPIN ALFA (BOVINE). Therefore, 50 μg and 100 μg doses are ineffective and 200 μg, 300 μg and 400 μg doses are effective. There were no significant differences in the percent viability among the three effective dose groups (Groups 3, 4, and 5). Overall, this study demonstrates that doses between 200 μg and 400 μg produced desirable results and these doses can be used for the purposes of superovulation in Bos taurus beef cows.

### Example 2: One Dose Treatment with a Bovine Follicle Stimulating Hormone Induces Superovulation in Mature Dairy Cows of Bos taurus Subtype

The study is a parallel arm, placebo-controlled study designed to measure the response of RIPAFOLLITROPIN ALFA (BOVINE) in dairy cows under conditions of field use. There are two treatment groups (saline negative control and 300 μg/cow) with 20 cows per group (groups may be enrolled in cohorts until full enrollment is reached).

TABLE 7
Treatment Summary

| | | | | | Treatment |
|---|---|---|---|---|---|
| Group | Number | Treatment | Regimen | Route | Time |
| A | 20 | CP: Saline | Once on | IM | Days 9-11 |
| | | | Day 0 | | of estrous |
| | | | | | cycle |
| B | 20 | IVP: 300 μg | Once on | IM | Days 9-11 |
| | | RIPAFOLLITROPIN | Day 0 | | of estrous |
| | | ALFA (BOVINE)/ | | | cycle |
| | | cow | | | |

### IVP

The composition of the formulation used is as in Example 1.

### Efficacy

Of the 20 cows treated with 300 μg of FSH, all 20 cows produced transferrable embryos (embryo quality codes 1+2+3 and developmental stage 3-9).

For the primary endpoint, the mean number of transferable embryos and their respective confidence limits are shown in Table 8. The mean transferable embryos was 7.25 and the lower bound of the 95% confidence limit was greater than 1. The mean transferrable embryos, along with the upper and lower 95% confidence limits, show that the 300 μg FSH was efficacious for superovulation in dairy cows, satisfying the primary efficacy criteria.

For the secondary endpoint for efficacy, the mean number of freezable embryos and their respective confidence limits are shown in Table 9. The mean transferable embryos was 6.03 and the lower bound of the 95% confidence limit was greater than 1. The mean freezable embryos, along with the upper and lower 95% confidence limits, show that the 300 μg FSH was efficacious for superovulation in dairy cows, satisfying the primary efficacy criteria for this secondary endpoint.

TABLE 8
Mean transferable embryos (standard error) recovered from cows treated
with 300 μg of rbFSH, with lower and upper confidence limits

| Treatment | Mean Transferable Embryos | 95% Confidence Limits | |
|---|---|---|---|
| (μg) | (Standard Error) | Lower | Upper |
| Placebo | 0.233 (0.663) | −1.1158 | 1.5825 |
| 300 | 7.25 (0.547) | 6.0816 | 8.4184 |

TABLE 9
Mean freezable embryos (standard error) recovered from cows treated
with 300 μg of rbFSH, with lower and upper confidence limits

| Treatment | Mean Freezable Embryos | 95% Confidence Limits | |
|---|---|---|---|
| (μg) | (Standard Error) | Lower | Upper |
| Placebo | .03333 | −1.3579 | 1.4246 |
| 300 | 6.025 | 4.8201 | 7.2299 |

Follicular stimulation and ovulation following treatment are measurements of FSH activity and were observed as secondary endpoints.

Follicular stimulation was measured by counting the number of medium and large follicles after treatment. The results are listed in Table 10A. The mean number of medium and large follicles after treatment with placebo and RIPAFOLLITROPIN ALFA (BOVINE) was 3.79 and 24.10, respectively. This demonstrates that ovaries from animals treated with RIPAFOLLITROPIN ALFA (BOVINE) had significantly higher stimulation as compared with ovaries from the placebo group.

Ovulation was measured by counting the number of corpora lutea on the ovaries on Day 12. The results are listed in Table 10B. The mean number of corpora lutea after treatment with placebo and RIPAFOLLITROPIN ALFA (BOVINE) was 0.867 and 15.00, respectively. This demonstrates a significantly higher number of ovulations per animal in the RIPAFOLLITROPIN ALFA (BOVINE) group, as compared with the placebo group.

TABLE 10A
Mean number of medium + large follicles (standard
error) measured on Day 3, after treatment on Day
0, with lower and upper confidence limits

| Treatment | Mean Medium + Large Follicles | 95% Confidence Limits | |
|---|---|---|---|
| (μg) | (Standard Error) | Lower | Upper |
| Placebo | 3.7895 | −0.3364 | 7.9154 |
| 300 | 24.1000 | 20.0786 | 28.1214 |

TABLE 10B
Mean number of corpora lutea (standard error) measured on Day 12,
after treatment on Day 0, with lower and upper confidence limits

| | | |
|---|---|---|
| Treatment | Mean Corpora Lutea | 95% Confidence Limits |

| (μg) | (Standard Error) | Lower | Upper |
|---|---|---|---|
| Placebo | 0.8667 | −1.0068 | 2.7401 |
| 300 | 15.0000 | 13.3775 | 16.6225 |

### Safety

The overall safety of RIPAFOLLITROPIN ALFA (BOVINE) for dairy cows was assessed by daily general health observations. No abnormal health observations were recorded for any cow during the course of the study.

### Conclusions

The results of this study show that a 300 μg dose of FSH was safe and efficacious when administered to dairy cows of Bos taurus subtype for superovulation.

### Example 3: Improved Method for Ovum Pick Up in Bos taurus Cows

This example reports an efficacy study of FSH for optimized oocyte recovery in Bos taurus cows. The study was designed to test the response to a single-chain recombinant FSH (RIPAFOLLITROPIN ALFA (BOVINE)) under conditions of field use to determine if and how single-chain FSH can improve oocytes recovery.

Three treatment groups (50 μg/cow, 100 μg/cow, and 200 μg/cow) with 20 cows in each group were used, as summarized below:

TABLE 11
Treatment Summary

| | Dosage of | | | |
|---|---|---|---|---|
| Treatment | RIPAFOLLITROPIN | | | |
| Group | ALFA (BOVINE) | N | Regimen | Route |
| A | 50 μg/cow | 20 | 1 ml on Day 0 | Intramuscular |
| B | 100 μg/cow | 20 | 2 ml on Day 0 | Intramuscular |
| C | 200 μg/cow | 20 | 4 ml on Day 0 | Intramuscular |

Cycling, mature Bos taurus beef cows, (e.g., Angus and Angus-cross) at least 13 months old, were used in the study. Cows with a normal physical examination, determined to be in estrus on Day −7 to Day −13, non-pregnant, and cycling normally as of Day −1 were enrolled.

### Treatment Administration

FSH was administered according to the treatment group assignment: Group A: 50 μg/cow (1 mL RIPAFOLLITROPIN ALFA (BOVINE)), Group B: 100 μg/cow (2 mL RIPAFOLLITROPIN ALFA (BOVINE)) and Group C: 200 μg/cow (4 mL RIPAFOLLITROPIN ALFA (BOVINE)) to all 20 animals allocated to each treatment group. Treatment was administered once, intramuscularly on Day 0 in the neck using a sterile disposable syringe fitted with a sterile disposable 1.5 inch 20 gauge needle. Each animal was treated with a new needle and syringe.

### Efficacy Parameters

On Day 3, an ultrasound examination is performed to evaluate the number and sizes of follicles (small=<3 mm, medium=3-7 mm and large ≥8 mm) on each ovary. Oocytes are then aspirated.

Oocyte recovery rate is defined as the percentage of oocytes per medium sized follicle.

Both follicle size (≥40% medium follicles, preferably above 50%) and recovery rate should preferably be met for a cow to be considered a treatment success.

Embryos are classified according to the following guidelines:
Grade 1—Excellent or Good (symmetrical and spherical embryos mass with individual blastomeres that are uniform in size, color and density). Grade 1 embryos are eligible to be frozen.
Grade 2—Fair quality (moderate irregularities in the overall shape of the embryo mass or in size, color and density of blastomeres). Grade 2 embryos are eligible to be frozen.
Grade 3—lower quality (irregularities in shape of the embryo mass or in size, color and density of the blastomeres). Grade 3 embryos are eligible to be transferred but cannot be frozen.

### Results

### Follicles Generated

The following tables summarize the % large vs small vs medium follicles in treated animals.
TRT: Treatment group
LSM: Least squares mean
SE: Standard error

TABLE 12
Percent small follicles

| TRT | LSM | SE |
|---|---|---|
| A | 67% | 4.0% |
| B | 33% | 4.0% |
| C | 15.5% | 4.0% |

TABLE 13
Percent medium follicles

| TRT | LSM | SE |
|---|---|---|
| A | 31.3% | 4.0% |
| B | 59.4% | 4.0% |
| C | 73.2% | 4.0% |

TABLE 14
Percent large follicles

| TRT | LSM | SE |
|---|---|---|
| A | 1.6% | 1.8% |
| B | 7.6% | 1.8% |
| C | 11.4% | 1.8% |

Remarkably, treatment groups B and C had high percentage of large follicles, as compared to treatment group A.

Treatment group A had a significantly high percentage of small size follicles. In contrast, treatments B and C had much less small size follicles.

Cows treated with Treatment A had the lowest percentage of medium follicles. Both cows on treatment B and C had more than 50% medium size follicles.

These data show treatments B and C induce follicles with most efficient size and profile.

TABLE 15
Recovery Rate

| TRT | LSM | SE |
|---|---|---|
| A | 68.2% | 2.9% |
| B | 67% | 3.0% |
| C | 64.6% | 3.0% |

These results show a very efficient recovery rate. Recovery rate is particularly efficient in group B which, in combination with the follicles profile, provide high efficacy treatment in OPU protocol.

TABLE 16
Embryo Development Rate

| TRT | LSM | SE |
|---|---|---|
| B | 64.9% | 4.0% |
| C | 66.1% | 4.0% |

These data show that the collected oocytes have a very high yield in subsequent embryo development.

This example demonstrates that optimal OPU can be achieved with a single 100 μg or 200 μg FSH injection. Considering oocyte recovery rate, 100 μg even appears most efficient.

### Example 4: Improved Method for Ovum Pick Up in Bos taurus Heifers

Studies were carried out to evaluate the effect of treatment with long-acting recombinant FSH on the efficiency of OPU in Holstein heifers. The effects of treatment on follicular diameter, viable oocyte rate and blastocyst rate were evaluated. Ninety Holstein heifers were used. On a random day of the estrous cycle, heifers received an intravaginal progesterone device (PRID) and 2 mg of estradiol benzoate plus PGF2á IM (Day 0). Heifers in the control group received no further treatment, whereas heifers in the r-FSH group received a single dose of 50 or 100 μg of rFSH on Day 4. On Day 7 the PRID was removed and OPU was conducted in all groups. The same operator aspirated all donors, and the same batch of sexed-semen was used for in vitro embryo production in all groups.

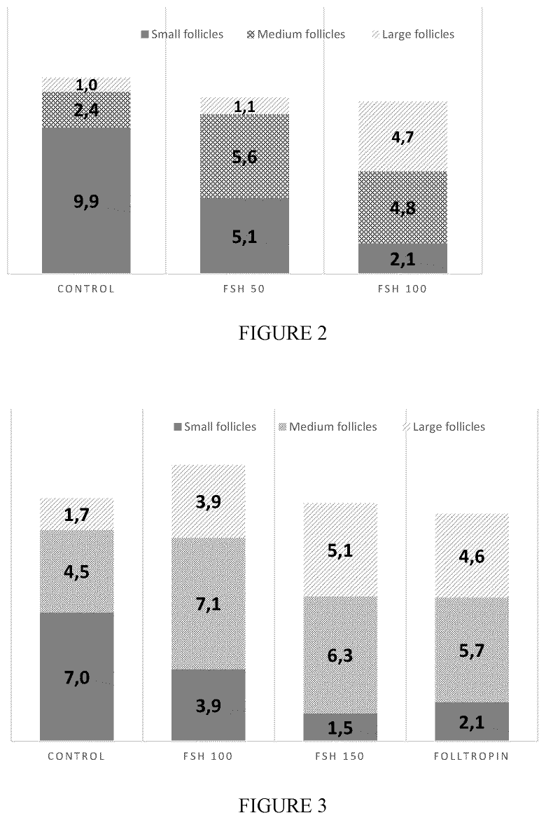

FIG. 2 shows the number of small, medium and large follicles in the heifers depending on the doses of rFSH administered. There was no difference between groups in the total numbers of follicles aspirated per OPU session (control=13.2+1.4 vs 50 μg=11.9+1.2 vs 100 μg=11.6+0.9; P=0.90). However, treatment with r-FSH regardless of dosage (50 or 100 μg) decreased the number of small follicles (<6 mm) and increased the number of medium follicles (6-10 mm) when compared with control group. Heifers that received 100 μg of r-FSH had a greater number of large follicles (<10 mm) at the time of OPU than control.

TABLE 17
Summary of OPU response (mean ± SEM) of
heifer donors (Holstein) submitted to different
doses of rFSH treatment previously to OPU.

| | Control | FSH 50 | FSH 100 |
|---|---|---|---|
| | (n = 30) | (n = 30) | (n = 30) |
| Viable oocytes rate, % | 54.0% | 62.0% | 71.0% |
| Total blastocysts, n | 0.97 ± 0.22 | 1.67 ± 0.40 | 1.37 ± 0.28 |
| Blastocyst rate, % | 7.1% | 16.2% | 17.4% |

Heifers treated with 100 μg of rFSH had a higher (P=0.004) viable oocytes rate than control group (control=54.0%^{b} vs 50 μg=62.0%^{ab} vs 100 μg=71.0%^{a}; P=0.004), without compromising the recovery rate.

Furthermore, heifers treated with 100 μg of rFSH had a higher cleavage (control=30.9%^{b} vs 50 μg=41.4%^{ab} vs 100 μg=52.3%^{a}; P=0.02) and blastocysts rates (control=7.1%^{b} vs 50 μg=16.2%^{ab} vs 100 μg=17.4%^{a}; P=0.05).

In conclusion, the treatment with a single dose of r-FSH increased the efficiency of OPU in Holstein heifers. The treatment increased the number of medium and large follicles at the time of aspiration, without compromising the recovery rate. In addition, it increased the viable oocytes rate, the cleavage rate and the blastocysts rate.

### Example 5: Improved Method for Ovum Pick Up in Lactating Bos taurus Cows

Studies were carried out to evaluate the effect of treatment with long-acting recombinant FSH on the efficiency of OPU in lactating Holstein donors. The effects of treatment on follicular diameter at OPU, viable oocyte rate and blastocyst rate were evaluated. One hundred twenty-two donors were used. On a random day of the estrous cycle, cows received an intravaginal progesterone device (PRID) and 2 mg of estradiol benzoate plus PGF24 IM (Day 0). Cows in the control group (n=31) received no further treatment, whereas cows in the r-FSH groups received a single dose of 100 μg (n=31) or 150 μg (n=33) of r-FSH on Day 4. Cows in the p-FSH group (Folltropin, Vetoquinol; n=27) received a total dosage of 200 mg of p-FSH on Days 4 and 5 in four decreasing doses 12 hours apart (57, 57, 43, and 43 mg). On Day 7 the PRID was removed and OPU was conducted in all groups. The same operator aspirated all donors, and the same batch of sexed-semen was used for in vitro embryo production in all groups.

FIG. 3 shows the number of small, medium and large follicles in lactating cows submitted to OPU with different doses of rFSH. There was no difference between groups (P=0.21) in the total numbers of follicles aspirated per OPU session (mean of 12.6+0.98). However, r-FSH (regardless of dosage; 100 or 150 μg) and p-FSH treatments decreased the number of small follicles (<6 mm) and increased the number of large follicles (<10 mm) at the time of OPU (P<0.0001) compared to control group.

TABLE 18
Summary of OPU response (mean ± SEM) of
lactating Holstein donors submitted to different
doses of rFSH treatment previously to OPU

| | Control | FSHr 100 | FSHr 150 | FOLLTROPIN |
|---|---|---|---|---|
| | (n = 31) | (n = 31) | (n = 33) | (n = 27) |
| Viable oocytes rate, % | 63.4% | 70.6% | 65.5% | 69.6% |
| Total blastocysts, n | 1.84 ± | 1.58 ± | 2.58 ± | 1.46 ± |
| | 0.30 | 0.30 | 0.39 | 0.25 |
| Blastocyst rate, % | 17.6% | 14.1% | 27.4% | 18.8% |

The blastocysts rate (number blastocysts/number oocytes cultured) was higher in donors treated with the 150 μg of r-FSH (27.4%) when compared to donors treated with 100 μg of r-FSH (14.1%; P=0.01).

Furthermore, the dose of 150 μg of r-FSH (2.58+0.39) showed a higher number of blastocysts when compared to the 100 μg of r-FSH (1.58+0.30) and p-FSH (1.46+0.25) groups (P=0.07).

### Example 6: Effect of rFSH Treatment on Quality of In Vitro Embryo Production

The P/ET (pregnancy rate) of in vitro produced embryos from Holstein donors (heifers, lactating cows, dry cows) treated with a single dose of 100 μg of r-FSH 3 days before OPU was also evaluated. Embryos were transferred into recipients (lactating Holstein cows) on the same day by the same operator.

TABLE 19
Pregnancy rate in recipients receiving in
vitro produced embryos from Holstein donors

| Category (donors) | Control | rFSH 100 |
|---|---|---|
| Heifer | 25.0% (7/28) | 31.1% (13/41) |
| Dry cow | 23.3% (14/60) | 32.1% (26/81) |
| Lactating cow | 32.1% (18/56) | 42.9% (21/49) |
| Total | 27.1% (39/144) | 35.1% (60/171) |

There was an increase in P/ET of recipients that received embryos from donors treated with rFSH [control: 27.1% (39/144) vs r-FSH 35.1% (60/171); P=0.06].

These data show that treatment with a single dose of rFSH before OPU increases not only the number of embryos produced per donor per OPU, but overall increases also the quality of those embryos, which translates into a higher level of pregnancy establishment and more live calves.

1. The use of a Follicule Stimulating Hormone (FSH), or of a composition comprising a FSH, to induce or stimulate superovulation in a Bos taurus cow, wherein said FSH comprises a bovine FSH alpha subunit and a bovine FSH beta subunit covalently linked to each other by a peptide linker, and wherein said FSH is administered once during estrous cycle to said Bos taurus cow at a dose comprised between 200 μg and 400 μg.

2. The use of claim 1, wherein said FSH is administered at a dose comprised between 250 μg and 350 μg.

3. The use of claim 1, wherein said FSH is administered at a dose of 300 μg.

4. The use of claim 1, wherein said FSH is administered by systemic administration.

5. The use of claim 1, wherein the alpha subunit is a polypeptide comprising an amino acid sequence that is at least 95% identical to SEQ ID NO: 1 and/or the beta subunit is a polypeptide comprising an amino acid sequence that is at least 95% identical to SEQ ID NO: 2.

6. The use of claim 1, wherein the FSH comprises an amino acid sequence having at least 95% identity to the amino acid sequence SEQ ID NO: 3.

7. The use of claim 6, wherein the FSH comprises SEQ ID NO: 3.

8. The use of claim 1, wherein the FSH is administered once between day 7 and day 12 of estrous cycle.

9. The use of claim 1, wherein the cows are synchronized.

10. The use of claim 1, further comprising inseminating said cow.

11. The use of claim 10, wherein insemination is performed 5 to 7 days after FSH administration.

12. The use of claim 1, comprising:
(a) providing a Bos taurus cow or a group of Bos taurus cows;
(b) administering to said cow or cows, during estrous cycle, one dose of about 300 μg of a FSH; and
(c) optionally inseminating the cow or cows of (b).

13. The use of claim 1, wherein said FSH is formulated in a composition with one or several excipients or carriers.

14. A method to increase pregnancy rate or insemination success in a Bos taurus cow, wherein the method comprises administering to said cow during estrous cycle a single dose of between 200 μg and 400 μg of a FSH.

15. A method according to claim 14, wherein the method comprises administering to said cow during estrous cycle a single dose of between 250 μg and 350 μg.

16. A method according to claim 14, wherein the method comprises administering to said cow during estrous cycle a single dose of 300 μg.
